(19) United States
(12) Patent Application Publication
(10) Pub. No.: US 2012/0065644 A1
(43) Pub. Date: Mar. 15, 2012

(54) STENT DEPLOYMENT SYSTEM WITH RETRACTABLE SHEALTH

(75) Inventors: Darren Ng, Mountain View, CA (US); Linda Thai, Los Angeles, CA (US)

(73) Assignee: Abbott Cardiovascular Systems, Inc., Santa Clara, CA (US)

(21) Appl. No.: 12/882,503

(22) Filed: Sep. 15, 2010

(51) Int. Cl. A61F 2/84; A61F 2/86

(52) U.S. Cl. 606/108; 623/1.11

(57) ABSTRACT

A stent deployment system is disclosed. The system has a stent positioned on the distal end of a delivery catheter and a retractable sheath covering the stent. The retractable sheath has one or more wires connected thereto to facilitate retraction of the sheath from off of the stent. The one or more wires extend from the sheath along a portion of the outer surface of the delivery catheter and then enter one or more lumens within the delivery catheter through which the one or more wires extend toward a retraction mechanism. The application of a proximally directed force upon the one or more wires causes the one or more wires and the sheath to move proximally relative to the delivery catheter and the stent, thereby uncovering the stent. Once uncovered, the stent can be expanded to provide scaffolding support a patient's luminal wall.

### BACKGROUND OF THE INVENTION

1. The Field of the Invention

The present invention generally relates to the delivery and deployment of stents within a body cavity. More particularly, the present invention relates to an improved and simplified stent delivery and deployment system that employs one or more wires to retract a sheath off of a stent for deployment of the stent within a body cavity.

2. The Relevant Technology

Stents, grafts, and a variety of other endoprostheses are well known and used in interventional procedures, such as for treating aneurysms, for lining or repairing vessel walls, for filtering or controlling fluid flow, and for expanding or scaffolding occluded or collapsed vessels. Such endoprostheses can be delivered and used in virtually any accessible body lumen of a human or animal. One recognized use of endoprostheses, such as stents, is for the treatment of atherosclerotic stenosis in blood vessels. For example, after a patient undergoes a percutaneous transluminal coronary angioplasty or similar interventional procedure, a stent is often deployed at the treatment site to improve the results of the medical procedure and to reduce the likelihood of restenosis. The stent is configured to scaffold or support the treated blood vessel. If desired, a stent can also be loaded with a beneficial agent so as to act as a delivery platform to reduce restenosis or for other beneficial purposes.

An endoprosthesis is typically delivered by a catheter delivery system to a desired location or deployment site inside a body lumen of a vessel or other tubular organ. To facilitate such delivery, the endoprosthesis and the delivery system can be capable of having a particularly small cross-sectional profile to access deployment sites within small diameter vessels. Additionally, the intended deployment site may be difficult to access by a physician and can involve traversing the delivery system through a tortuous luminal pathway. Thus, it can be desirable to provide the delivery system and endoprosthesis with a sufficient degree of flexibility during delivery to allow advancement through the anatomy to the deployed site.

Various systems and devices have been developed for delivering and implanting endoprosthesis, such as stent, grafts, and the like, within a body lumen. For instance, one method includes compressing or crimping a stent to reduce its diameter. The stent is mounted on the distal end of a delivery catheter and a tubular sheath is placed over the stent. The sheath can act as a restraint to maintain the stent in the compressed or crimped configuration, particularly in the case of a self-expanding stent. The sheath can also provide protection to the stent, regardless of whether the stent is a self-expanding stent or a balloon-expandable stent. The sheathed stent can then be delivered to the desired location within the patient. This can be accomplished by passing the distal end of the delivery catheter and the sheathed stent through a guide catheter that is positioned within the patient's vasculature. When the stent has been delivered to the desired location within the patient, the sheath can be removed to uncover the stent. With the sheath removed, the stent can be expanded to provide scaffolding support to the walls of the body lumen. In the case of a balloon-expandable stent, the stent can be expanded by inflating a balloon upon which the stent is mounted. In the case of a self-expanding stent, simply removing the sheath from the stent can allow the stent to radially expand against the lumen walls.

Typically, the stent is mounted on the distal end of the delivery catheter and the proximal end of the delivery catheter is attached to a manipulator handle that remains outside the patient's body Likewise, the sheath is attached to the manipulator handle by way of a continuous catheter that is disposed over the delivery catheter. The sheath is removed off of the stent by actuating a mechanism on the manipulator handle, such as a thumb wheel, which is hand operated by the physician. When the thumb wheel is operated, the continuous catheter is proximally withdrawn relative to the stent, delivery catheter, and guide catheter. Since the distal end of the continuous catheter is attached to the sheath, proximally withdrawing the continuous catheter also proximally withdraws the sheath relative to the stent, delivery catheter, and guide catheter, thereby uncovering the stent and allowing for radial expansion of the stent.

Problems can arise when the sheath and the continuous catheter are retracted proximally by the application of a pulling force. For instance, friction between the delivery catheter and the continuous catheter/sheath must be overcome by the pulling force in order for the stent to be uncovered. The tensile force exerted on the continuous catheter and the sheath will be opposed by an equivalent compressive force exerted on the deliver catheter. When longitudinal compression of the deliver catheter occurs, the sheath may not retract completely or at all relative to the stent. Leaving the stent partially or entirely covered by the sheath can partially or entirely prevent the stent from radially expanding.

In order to avoid longitudinal compression of the delivery catheter when the sheath is being retracted, various types of stabilizers have been used. Some stabilizers include such things as a metal hypotube that can be positioned around and attached to the delivery catheter to reinforce and increase the compression resistance of the delivery catheter. Although a hypotube can help a delivery catheter resist compression, hypotubes are typically not flexible enough to allow for the delivery catheter to navigate though tortuous luminal passages. In other cases, longitudinal compression of the delivery catheter is avoided by making the delivery catheter thicker and stiffer. As with the hypotubes, thick and stiff delivery catheters can be difficult to maneuver through narrow, tortuous luminal passages.

Accordingly, it would be desirable to have a stent deployment system that includes a retractable sheath for selectively uncovering a stent, and which allows for the retraction of the sheath while avoiding significant longitudinal compression of a delivery catheter without the use of bulky delivery catheters or additional stabilizers. The subject matter described and claimed herein is not limited to embodiments that solve any disadvantages or that operate only in environments such as those described above. Rather, this background is provided to illustrate one exemplary technology area where some embodiments described herein may be practiced.

### BRIEF SUMMARY OF THE INVENTION

The present invention relates to an improved and simplified stent delivery and deployment system that employs one or more wires to retract a sheath off of a stent for deployment of the stent within a body cavity. Embodiments of the present invention can reduce the likelihood of longitudinal compression of a delivery catheter which can lead to incomplete retraction of a sheath and/or deployment of a stent away from the desired treatment site.

In one aspect of the invention, there is provided a stent deployment system for delivering and deploying a stent within a body lumen. The system includes a delivery catheter, a sheath, and at least one wire attached to the sheath. The delivery catheter has a proximal end and a distal end, with the distal end being able to have a stent disposed thereon for delivery into a body lumen. The delivery catheter also has at least one slot that extends from an outer surface to an interior portion of the delivery catheter. The interior portion can include a guide wire lumen or a separate retraction wire lumen. The sheath can be disposed or positioned around the distal end of the delivery catheter to selectively cover the stent. The sheath can also move along a length of the delivery catheter to selectively uncover the stent. The distal end of the at least one wire is attached to the sheath, such as to the inner surface thereof, to facilitate movement of the sheath so that the stent can be selectively uncovered. The at least one wire passes from the outer surface of the delivery catheter to the interior portion of the delivery catheter through the at least one slot in the delivery catheter.

According to another exemplary embodiment of the invention, a stent deployment system includes a delivery catheter that has a proximal end, a distal end, an intermediate portion extending therebetween. The delivery catheter also has a guide wire lumen extending therethrough. The distal end of the delivery catheter can have a stent disposed thereon. The intermediate portion of the delivery catheter includes at least one slot that extends from an outer surface to the guide wire lumen. The system also includes a sheath adapted to be disposed around the distal end of the delivery catheter to cover the stent. The sheath can move along a length of the delivery catheter to selectively uncover the stent. The distal end of at least one wire is attached to the sheath to facilitate movement of the sheath to selectively uncover the stent. The at least one wire passes from the outer surface of the delivery catheter into the guide wire lumen through the at least one slot in the delivery catheter.

According to still yet another exemplary embodiment of the present invention, a stent deployment system is provided for delivering and deploying a stent within a body lumen. The system includes a delivery catheter, a sheath, and at least one wire attached to the sheath. The delivery catheter has a proximal end, a distal end, and an intermediate portion extending therebetween. A stent can be positioned on the distal end of the delivery catheter. The delivery catheter also includes at least one retraction wire lumen disposed therein. The retraction wire lumen extends from an opening in an outer surface of the intermediate portion to the proximal end of the delivery catheter. The sheath can be positioned around the distal end of the delivery catheter to selectively cover the stent. The sheath can also be moved along the length of the delivery catheter to selectively uncover the stent. The at least one wire that is attached to the sheath facilitates the movement of the sheath to selectively uncover the stent. The at least one wire extends from the sheath, into the opening in the outer surface of the intermediate portion, and through the at least one retraction wire lumen toward the proximal end of the delivery catheter.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an elevational view of a stent delivery system according to one exemplary embodiment of the present invention;

FIG. 2 is an elevational view of the stent delivery system of FIG. 1 with a sheath in a withdrawn position to expose a stent;

FIG. 3 is a cross-sectional view of a portion of a stent delivery system according to an exemplary embodiment of the present invention;

FIG. 4 is a cross-sectional view of the portion of the stent delivery system of FIG. 3 showing a sheath in a withdrawn position to expose a stent;

FIG. 5 is a cross-sectional view of a portion of a stent delivery system according to another exemplary embodiment of the present invention; and

FIG. 6 is a cross-sectional view of the portion of the stent delivery system of FIG. 5 showing a sheath in a withdrawn position to expose a stent.

### DETAILED DESCRIPTION OF THE PREFERRED EMBODIMENTS

Reference will now be made to the drawings to describe various aspects of exemplary embodiments of the invention. It is understood that the drawings are diagrammatic and schematic representations of such exemplary embodiments, and are not limiting of the present invention, nor are they necessarily drawn to scale. In the following description, numerous specific details are set forth in order to provide a thorough understanding of the present invention. It will be obvious, however, to one of ordinary skill in the art that the present invention may be practiced without these specific details. In other instances, well-known aspects of stents, catheters, guide wires, stent deployment systems, and the like, have not been described in particular detail in order to avoid unnecessarily obscuring the present invention.

As used herein, the terms “proximal,” “proximally,” and “proximal direction” are intended to mean positioned, disposed, or moving away from or out of the patient, and the terms “distal,” “distally,” and “distal direction” are intended to mean positioned, disposed, moving toward or into the patient.

As shown in the exemplary drawings, the present invention includes a stent deployment system for delivering and deploying a stent within a body lumen, such as a vessel. Frequently, after balloon angioplasty has been performed to dilate a stenosis in the lumen of a vessel, a stent is deployed at the treated site to aid in healing and to prevent restenosis. Typically, a stent is delivered and deployed by first compressing the stent, mounting the stent at the distal end of a delivery catheter, and slidably disposing the delivery catheter and stent within the lumen of a sheath to hold the stent in a contracted condition. Once the delivery catheter and stent are advanced to the desired location within the body lumen (the “treatment site”), the sheath is retracted to expose the stent, thereby allowing for radial expansion of the stent against the lumen wall. In the case of a self-expanding stent, retracting the sheath off of the stent allows for the stent to self-expand. With a balloon-expandable stent, retracting the sheath off of the stent allows for the stent to be expanded by inflating a balloon upon which the stent is mounted.

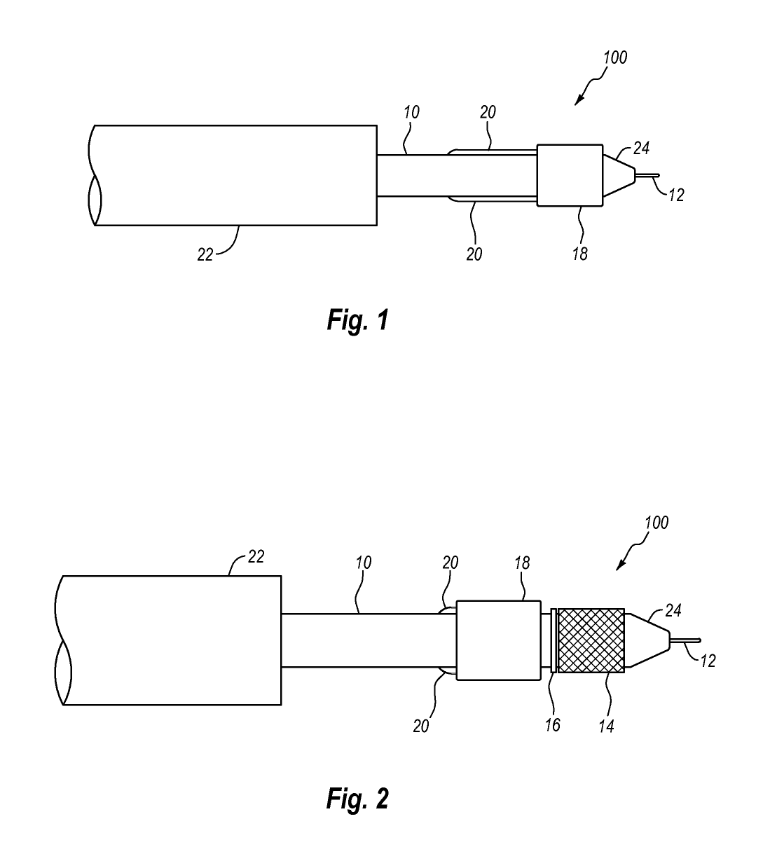

FIGS. 1-6 illustrate exemplary stent deployment systems that embody features of the present invention. In the elevational views of FIGS. 1-2, a stent deployment system 100 according to one exemplary embodiment of the present invention is illustrated as having a delivery catheter 10 with a guide wire lumen (not shown) therethrough adapted to receive a guide wire 12. Delivery catheter 10 has proximal and distal ends. As seen in FIG. 2, a stent 14 can be mounted on the distal end of delivery catheter 10. Stent 14 can be either a self-expanding stent or a balloon expandable stent. Stents are known in the art and stent 14 can be of any suitable design. It will be appreciated, however, that endoprostheses other than stents can be utilized in connection with the present invention.

Stent delivery system 100 also optionally includes a tip 24 attached to the distal end of delivery catheter 10. Including a tip, such as tip 24, can provide various benefits. For instance, a soft, generally cone shaped tip can facilitate smooth advancement of stent delivery system 100 through a patient's vasculature by preventing a snow plowing effect that may be experienced without such a tip. Tip 24 can be made from any suitable material, including a polymeric material. Tip 24 can also include a radiopaque element that provides a visualization reference point for a physician during fluoroscopy. As is known, there are a variety of tips used at the end of delivery catheters, any of which could be used in connected with the present invention.

As illustrated in FIG. 2, a stop 16 is optionally fixedly mounted or formed on delivery catheter 10 near the distal end of delivery catheter 10. Stop 16 can be formed or mounted on delivery catheter 10 to limit or prevent stent 14 from moving proximally relative to or along the length of delivery catheter 10. Stop 16 can be an annular protrusion, a simple projection, a ring securely mounted on delivery catheter 10, or the like, to limit or prevent proximal movement of stent 14.

Once stent 14 has been crimped or compressed and mounted on the distal end of delivery catheter 10 as shown in FIG. 2, a sheath 18 can be placed over stent 14 to cover stent 14 as shown in FIG. 1. Sheath 18 can provide protection to stent 14 and/or maintain stent 14 in the crimped or compressed configuration until stent 14 is delivered to the treatment site within a patient. In the illustrated embodiment, sheath 18 is generally tubular in shape and can be slidably disposed over stent 14 when stent 14 is mounted in the crimped or compressed configuration on the distal end of delivery catheter 10. As discussed in greater detail below, sheath 18 can be operatively linked, via one or more wires 20, to a retraction mechanism (not shown), such as a manipulator handle, at the proximal end of stent deployment system 100 to enable the proximal retraction or withdrawal of sheath 18 relative to stent 14 and delivery catheter 10 to uncover stent 14.

FIG. 1 shows stent deployment system 100 assembled, arranged, or otherwise configured for advancement through a patient's vasculature. More particularly, when stent deployment system 100 is arranged in the delivery configuration shown in FIG. 1, stent 14 (not shown in FIG. 1) is mounted on the distal end of delivery catheter 10 in a crimped or compressed configuration (as shown in FIG. 2) and sheath 18 is disposed on or positioned around stent 14 to cover at least a portion of stent 14.

With stent deployment system 100 in the delivery configuration shown in FIG. 1, stent deployment system 100 can be advanced through a patient's vasculature to the treatment site. Typically, stent deployment system 100 is advanced to the treatment site with the aid of a guide catheter 22 and/or guide wire 12. By way of example, guide catheter 22 can be introduced into the vasculature of a patient through the femoral artery. After introduction, guide catheter 22 can be advanced through the vasculature until the distal end of guide catheter 22 is positioned just proximal to the treatment site. Stent deployment system 100 can then be advanced to the treatment site through guide catheter 22. Guide wire 12 can be slidably disposed within the guide wire lumen (not shown) of delivery catheter 10 to assist in guiding delivery catheter 10 through guide catheter 22 to the treatment site.

Once stent deployment system 100 reaches the distal end of guide catheter 22, guide wire 12 can be extended out from the distal end of delivery catheter 10 and advanced to the treatment site. Thereafter, the distal end of delivery catheter 10 (with stent 14 mounted or disposed thereon) can be advanced over guide wire 12 until stent 14 is positioned at the treatment site. With stent 14 positioned at the treatment site as desired, sheath 18 can be proximally retracted or withdrawn to the position shown in FIG. 2 to uncover stent 14. As discussed in greater detail below, sheath 18 can be retracted by applying a proximally directed force on wires 20. Since the distal ends of wires 20 are connected to sheath 18, the proximally directed force applied on wires 20 will also be applied, at least indirectly, to sheath 18. This proximally directed force will cause sheath 18 to be retracted in the proximal direction, thereby uncovering stent 14. With sheath 18 retracted and stent 14 exposed, stent 14 can be expanded against the lumen walls at the treatment site, either through self expansion or balloon expansion.

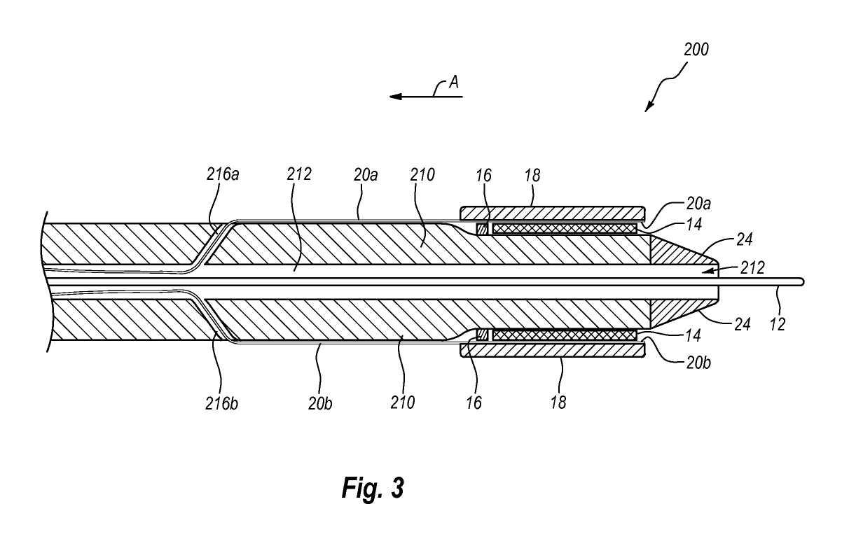

Attention is now directed to FIGS. 3-7, which illustrate exemplary embodiments of how sheath 18 can be linked to a retraction mechanism (not shown). The illustrated embodiments can provide various advantages over other stent deployment systems. As will be discussed, for instance, the illustrated embodiments include one or more wires that connect the sheath to a retraction mechanism. The relatively small surface area of the one or more wires creates relatively little friction as the wires move against the delivery catheter and/or the guide catheter when the sheath is retracted. With relatively little friction created between the wires and the delivery catheter, there is less likely to be longitudinal compression of the delivery catheter, which can improve the likelihood that the stent will be deployed at the desired location. Additionally, connecting the sheath to the retraction mechanism with one or more wires can use less material and be less costly than other types of devices used to connect a sheath to a retraction mechanism.

In contrast to the illustrated embodiments, other stent deployment systems connect the sheath to the retraction mechanism with a continuous catheter that is disposed around a large portion of the delivery catheter's length. A relatively significant amount of friction is created when the relatively large surface area presented by the continuous catheter rubs or moves against the delivery catheter as the continuous catheter is proximally retracted to remove the sheath from the stent. As discussed elsewhere herein, the friction between the continuous catheter and the delivery catheter can cause longitudinal compression of the delivery catheter. Longitudinal compression of the delivery catheter can partially or entirely prevent the sheath from being removed off of the stent and/or can cause the stent to move and/or be deployed away from the treatment site.

As a result of the problems associated with the friction between the continuous catheter and the delivery catheter, significant time and resources have been spent developing stabilizers that improve the column strength of the delivery catheter and prevent longitudinal compression of the delivery catheter and the problems associated therewith, such as incomplete retraction of the sheath and deployment of the stent away from the treatment site. Embodiments of the present invention provide a stent deployment system that enables retraction of the sheath without creating significant friction with the delivery catheter. As a result, the need for a stabilizer to resist longitudinal compression of the delivery catheter is reduced or eliminated.

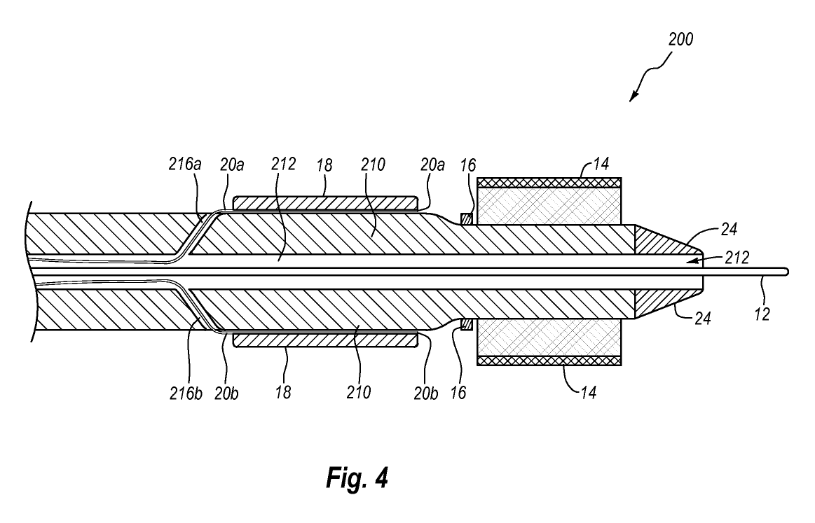

With specific reference to FIGS. 3-4, there is shown a longitudinal cross-section of an exemplary embodiment of the present invention. The stent deployment system 200 illustrated in FIGS. 3-4 is similar in many respects to stent deployment system 100 of FIGS. 1-2. For instance, stent deployment system 200 includes a delivery catheter 210 that has a stent 14 mounted on a distal end thereof. Delivery catheter 210 also has an optional stop 16 formed or mounted at the distal end thereof, proximal to stent 14, to limit or prevent the proximal movement of stent 14 relative to delivery catheter 210. Attached to the distal end of delivery catheter 210 is an optional tip 24. A guide wire lumen 212 extends through at least a portion of the length of delivery catheter 210 and through tip 24. As discussed above, a guide wire 12 can extend through guide wire lumen 212 to facilitate the advancement of stent deployment system 200 through the vasculature of a patient so that stent 14 can be deployed at the treatment site.

Like stent deployment system 100, stent deployment system 200 also includes a retractable sheath 18 for covering stent 14 in the contracted delivery configuration shown in FIG. 3. As discussed above, sheath 18 can provide protection to stent 14 during insertion and advancement through a patient's vasculature. When stent 14 is a self-expanding stent, sheath 18 can also act as a restraint to maintain stent 14 in the contracted delivery configuration shown in FIG. 3.

As can be seen in FIGS. 3-4, sheath 18 has two wires 20a, 20b attached thereto and which extend proximally along the length of delivery catheter 210. In the illustrated embodiment, the distal ends of wires 20a, 20b are attached to an interior surface of sheath 18. As shown, wires 20a, 20b can be attached along the entire length of sheath 18, between proximal and distal ends of sheath 18. Wires 20a, 20b can be attached to sheath 18 in any suitable manner, including with adhesives, such as glues, thermal or chemical bonds, or mechanical fasteners. In alternative embodiments, wires 20a, 20b may not be attached along the entire inner surface of sheath 18. For instance, wires 20a, 20b can be attached at the proximal end, the distal end, and/or somewhere between the proximal and distal ends of sheath 18. Similarly, wires 20a, 20b can also be coupled to the ends or outer surface of sheath 18 rather than to the inner surface as shown. As mentioned above, the proximal ends of wires 20a, 20b are attached or otherwise operatively associated with a retraction mechanism (not shown) that remains positioned outside the patient's body. Wires 20a, 20b, therefore, connect sheath 18 to a retraction mechanism outside the patient's body.

Once delivery catheter 210 and stent 14 are desirably positioned and secured in place at the treatment site, as discussed above, the retraction mechanism can be activated to pull wires 20a, 20b proximally (e.g., in the direction of arrow A) relative to delivery catheter 210 and stent 14. Since wires 20a, 20b are attached to sheath 18, the proximal movement of wires 20a, 20b also causes sheath 18 to move proximally relative to delivery catheter 210 and stent 14. Thus, upon activation of the retraction mechanism, sheath 18 is proximally retracted or withdrawn from the delivery position shown in FIG. 3 to the deployment position shown in FIG. 4. As can be seen in FIG. 4, when sheath 18 has been retracted to the deployment position, stent 14 is uncovered and is able to radially expand to provide scaffolding support to the patient's luminal wall.

With continued reference to FIGS. 3-4, it is noted that delivery catheter 210 also includes two slots, channels, lumens, or holes, such as slots 216a, 216b, that extend from an exterior surface of delivery catheter 210 to guide wire lumen 212. Slots 216a, 216b are configured and adapted to have wires 20a, 20b, respectively, passed therethrough. That is, for example, wire 20a extends proximally from the proximal end of sheath 18 along the outer surface of delivery catheter 210 to slot 216a, at which point wire 20a passes through slot 216a and into guide wire lumen 212. Upon entry into guide wire lumen 212, wire 20a extends proximally through guide wire lumen 212 toward the retraction mechanism. Similarly, wire 20b extends proximally from the proximal end of sheath 18 along the outer surface of delivery catheter 210 to slot 216b, at which point wire 20b passes through slot 216b and into guide wire lumen 212. Upon entry into guide wire lumen 212, wire 20b extends proximally through guide wire lumen 212 toward the retraction mechanism. Thus, as wires 20a, 20b are pulled proximally to move sheath 18 from the delivery configuration shown in FIG. 3 to the deployment configuration shown in FIG. 4, at least a portion of wires 20a, 20b that were disposed on the outer surface of delivery catheter 210 in the delivery configuration enter into slots 216a, 216b, respectively. As can be seen, wires 20a, 20b can then extend through guide wire lumen 212 generally parallel to guide wire 12.

A comparison between FIGS. 3 and 4 illustrates that slots 216a, 216b are located in delivery catheter 210 sufficiently far away from the distal end of delivery catheter 210 to allow sheath 18 to be completely retracted off of stent 14. As shown in FIG. 3, wires 20a, 20b extend from the proximal end of sheath 18 along an outer surface of delivery catheter 210 before entering into slots 216a, 216b. The length of the outer surface of delivery catheter 210 along which wires 20a, 20b extend is at least the length between the proximal and distal ends of sheath 18. Stated another way, the distance between slots 216a, 216b and the proximal end of stent 14 is at least the same or greater than the distance between the proximal and distal ends of sheath 18.

Slots 216a, 216b are so located to enable sheath 18 to be completely removed off of stent 14 as shown in FIG. 4. As can be seen in FIG. 4, when sheath 18 is retracted off of stent 14, the proximal end of sheath 18 is adjacent slots 216a, 216b while the distal end of sheath 18 is disposed proximally relative to the proximal end of stent 14 such that sheath 18 does not overlie or cover stent 14. It will be appreciated that slots 216a, 216b can be located even further from stent 14 than illustrated. In any case, slots 216a, 216b are located so that sheath 18 can be located between slots 216a, 216b and the proximal end of stent 14 so that stent 14 can be completely uncovered when sheath 18 is retracted. Once sheath 18 has been retracted as shown in FIG. 4, stent 14 can be expanded as shown. The expansion of stent 14 can be through self-expansion or balloon-expansion.

Using wires 20a, 20b to connect sheath 18 to a retraction mechanism, and thereby enable retraction of sheath 18 off of stent 14, provides various benefits. For instance, as wires 20a, 20b are retracted, relatively little friction is created between wires 20a, 20b and delivery catheter 210. Since wires 20a, 20b have relatively small surface areas, there will be less surface-to-surface contact between wires 20a, 20b and delivery catheter 210 compared to the amount of surface-to-surface contact between a continuous catheter and a delivery catheter typically used in stent deployment systems. This smaller amount of surface-to-surface contact can understandably reduce the amount of friction created between wires 20a, 20b and delivery catheter 210. As will be appreciated by one or ordinary skill in the art in light of the disclosure herein, minimizing or limiting the amount of friction between the components of a stent delivery system can reduce the likelihood of longitudinal compression of a delivery catheter and the challenges associated therewith.

As noted elsewhere herein, longitudinal compression of a delivery catheter (primarily caused by the friction created as a continuous catheter is pulled over the delivery catheter to retract the sheath) can prevent a sheath from being completely or entirely retracted off of a stent. Longitudinal compression of a delivery catheter can also cause the stent to be deployed away from the desired treatment site. That is, after the stent has been properly positioned at the treatment site and the continuous catheter is pulled back over the delivery catheter to remove the sheath, the longitudinal compression of the delivery catheter (caused by the friction between the continuous catheter and the delivery catheter) can move the stent away from the treatment site. Thus, even if the sheath is removed from the stent, the stent may be deployed away from the treatment site. Therefore, connecting sheath 18 to a retraction mechanism with one or more wires rather than a continuous catheter, as described herein, can avoid the challenges commonly encountered with the use of other stent deployment systems.

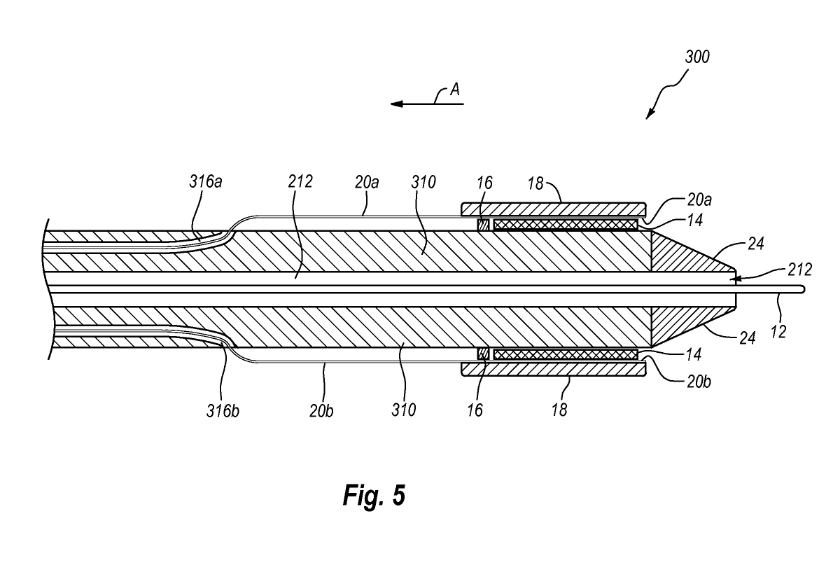

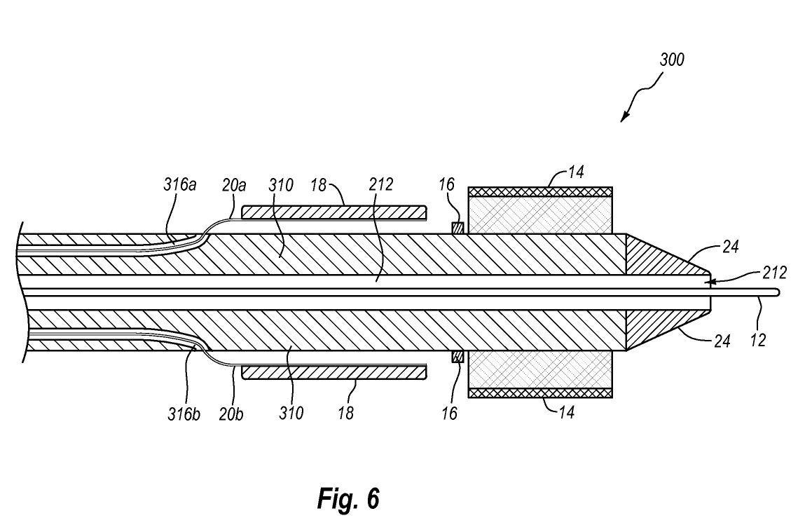

Attention is now directed to FIGS. 5-6, which illustrate a longitudinal cross-section of another exemplary embodiment of the present invention. The stent deployment system 300 illustrated in FIGS. 5-6 is substantially the same as stent deployment system 200 from FIGS. 3-4. Thus, for instance, stent deployment system 300 includes a delivery catheter 310 that has a stent 14 mounted on a distal end thereof. Delivery catheter 310 also has an optional stop 16 formed or mounted at the distal end thereof, proximal to stent 14, to limit or prevent the proximal movement of stent 14 relative to delivery catheter 310. Attached to the distal end of delivery catheter 310 is an optional tip 24. A guide wire lumen 212 extends through at least a portion of the length of delivery catheter 310 and through tip 24. As discussed above, a guide wire 12 can extend through guide wire lumen 212 to facilitate the advancement of stent deployment system 200 through the vasculature of a patient so that stent 14 can be deployed at the treatment site.

Stent deployment system 300 also includes a retractable sheath 18 for covering stent 14 in the contracted delivery configuration shown in FIG. 5. As discussed above, sheath 18 can provide protection to stent 14 during insertion and advancement through a patient's vasculature. When stent 14 is a self-expanding stent, sheath 18 can also act as a restraint to maintain stent 14 in the contracted delivery configuration shown in FIG. 5.

Also like stent delivery system 200, stent deployment system 300 also includes a sheath 18 that has two wires 20a, 20b attached thereto. As discussed above, wires 20a, 20b can be attached to the inner surface, the outer surface, or the proximal or distal ends of sheath 18, or a combination thereof, with any suitable attachment means, such as adhesives, thermal or chemical bonds, or mechanical fasteners. Wires 20a, 20b extend proximally along the length of delivery catheter 210. The proximal ends of wires 20a, 20b are attached or otherwise operatively associated with a retraction mechanism (not shown) that remains positioned outside the patient's body. Wires 20a, 20b, therefore, connect sheath 18 to a retraction mechanism outside the patient's body.

The main distinction between stent deployment system 200 and stent deployment system 300 is the difference between the slots formed in the delivery catheters for receiving the wires that are connected to the sheaths. As discussed above, stent deployment system 200 includes slots 216a, 216b which enable wires 20a, 20b to pass from an outer surface of delivery catheter 210 into guide wire lumen 212 so that wires 20a, 20b extend proximally through guide wire lumen 212 toward a retraction mechanism.

In contrast to slots 216a, 216b, stent delivery system 300 includes retraction wire lumens 316a, 316b. Retraction wire lumens 316a, 316b are similar to slots 216a, 216b in that retraction wire lumens 316a, 316b receive wires 20a, 20b therein so that wires 20a, 20b extend proximally through an interior portion of delivery catheter 310 toward a retraction mechanism. Retraction wire lumens 316a, 316b can, therefore, be considered to be slots, channels, lumens, or holes. Nevertheless, retraction wire lumens 316a, 316b do not open into or enable passage of wires 20a, 20b into guide wire lumen 212. Thus, the portion of delivery catheter 310 that has guide wire lumen 212 and retraction wire lumens 316a, 316b can be considered a tri-lumen catheter while the portion of delivery catheter 310 distal to retraction wire lumens 316a, 316b that only includes guide wire lumen 212 can be considered a single lumen catheter. In this manner stent delivery system 300 maintains wires 20a, 20b separate from one another and from guide wire 12.

According to the illustrated embodiment, when stent deployment system 300 is in a delivery configuration as shown in FIG. 5, wires 20a, 20b extend from the proximal end of sheath 18 and track along an outer surface of delivery catheter 310 until they reach retraction wire lumens 316a, 316b. Wires 20a, 20b then enter retraction wire lumens 316a, 316b and continue to extend proximally therethrough toward a retraction mechanism (not shown). Once stent 14 has been desirably positioned at the treatment site, sheath 18 can be retracted to the position shown in FIG. 6. More specifically, with stent 14 properly positioned at the treatment site, a retraction mechanism can be activated by a physician to pull wires 20a, 20b in the proximal direction (e.g., in the direction of arrow A) relative to delivery catheter 310 and stent 14. Since wires 20a, 20b are connected to sheath 18, movement of wires 20a, 20b in the proximal direction will cause sheath 18 to also move proximally relative to delivery catheter 310 and stent 14, thereby uncovering stent 14 as shown in FIG. 6. As wires 20a, 20b are pulled proximally, at least a portion of wires 20a, 20b that were disposed on the outer surface of delivery catheter 310 enter into retraction wire lumens 316a, 316b, respectively.

A comparison between FIGS. 5 and 6 illustrates that retraction wire lumens 316a, 316b are located in delivery catheter 310 sufficiently far away from the distal end of delivery catheter 310 to allow sheath 18 to be completely retracted off of stent 14. As shown in FIG. 5, wires 20a, 20b extend from the proximal end of sheath 18 along an outer surface of delivery catheter 310 before entering into retraction wire lumens 316a, 316b. The length of the outer surface of delivery catheter 310 along which wires 20a, 20b extend is at least the length between the proximal and distal ends of sheath 18. Stated another way, the distance between retraction wire lumens 316a, 316b and the proximal end of stent 14 is at least the same or greater than the distance between the proximal and distal ends of sheath 18.

Retraction wire lumens 316a, 316b are so located to enable sheath 18 to be completely removed off of stent 14 as shown in FIG. 6. As can be seen in FIG. 6, when sheath 18 is retracted off of stent 14, the proximal end of sheath 18 is adjacent retraction wire lumens 316a, 316b while the distal end of sheath 18 is disposed proximally relative to the proximal end of stent 14 such that sheath 18 does not overlie or cover stent 14. It will be appreciated that retraction wire lumens 316a, 316b can be located even further from stent 14 than illustrated. In any case, retraction wire lumens 316a, 316b are located so that sheath 18 can be located between retraction wire lumens 316a, 316b and the proximal end of stent 14 so that stent 14 is completely uncovered when sheath 18 is retracted. Once sheath 18 has been retracted as shown in FIG. 6, stent 14 can be expanded as shown. The expansion of stent 14 can be through self-expansion or balloon-expansion.

In addition to the benefits discussed above with regard to connecting sheath 18 to a retraction mechanism using wires 20a, 20b, the present embodiment also substantially prevents wires 20a, 20b from becoming tangled with one another or with guide wire 12. This is due to the fact that wires 20a, 20b extend through separate retraction wire lumens and guide wire 12 extends through guide wire lumen 212.

The components of the above-described stent deployment systems can be formed from any suitable materials. By way of non-limiting example, the delivery catheters described herein can be made of conventional polyethylene tubing, polyethylene (“PE tube”), or engineering polymers such as nylon, PEEK (polyethylene ethyl ketone) or PET (polyethylene terephthalate) Likewise, the components can be formed in any suitable manner. For instance, the delivery catheters can be formed through extrusion processes, braiding processes, or the like.

Additionally, it will be appreciated that the embodiments described herein can be modified without departing from the scope of the present invention. By way of non-limiting example, although the embodiments herein have been described as having two wires 20a, 20b and corresponding slots 216a, 216b or retraction wire lumens 316a, 316b, it will be appreciated that sheath 18 can be connected to a retraction mechanism with one or more wires 20 which extend into a guide wire lumen through one or more slots 216 or extend through one or more retraction wire lumens 316. Similarly, while wires 20a, 20b and corresponding slots 216a, 216b and retraction wire lumens 316a, 316b have been illustrated on opposing sides of sheath 18 and the delivery catheters, the one or more wires 20, slots 216, and/or retraction wire lumens 316 can be disposed in any desirable arrangement around the circumferences of sheath 18 and/or the delivery catheters.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A stent deployment system for delivering and deploying a stent within a body lumen, comprising:
a delivery catheter having a proximal end and a distal end, said distal end being adapted to have a stent disposed thereon for delivery into a body lumen, said delivery catheter also having at least one slot disposed therein that extends from an outer surface of said delivery catheter to an interior portion of said delivery catheter;
a sheath adapted to be disposed around said distal end of said delivery catheter to selectively cover the stent disposed on said distal end of said delivery catheter, said sheath being adapted for movement along a length of said delivery catheter to selectively uncover the stent; and
at least one wire having a distal end attached to said sheath to facilitate movement of said sheath along the length of said delivery catheter to selectively uncover the stent, said at least one wire passing from said outer surface of said delivery catheter to said interior portion of said delivery catheter through said at least one slot in said delivery catheter.

2. A stent deployment system as recited in claim 1, wherein said interior portion of said delivery catheter comprises a guide wire lumen adapted to have a guide wire passed therethrough.

3. A stent deployment system as recited in claim 2, wherein said at least one slot disposed in said delivery catheter extends from said outer surface of said delivery catheter to said guide wire lumen to enable said at least one wire to pass from said outer surface of said delivery catheter into said guide wire lumen.

4. A stent deployment system as recited in claim 1, wherein said at least one slot has a first end that opens to said outer surface of said delivery catheter at a location between said proximal and distal ends of said delivery catheter.

5. A stent deployment system as recited in claim 4, wherein said at least one slot further comprises a second end and an intermediate portion that extends between said first and second ends, said second end being disposed at said proximal end of said delivery catheter

6. A stent deployment system as recited in claim 5, wherein said delivery catheter further comprises a guide wire lumen extending between said proximal and distal ends thereof, said intermediate portion of said at least one slot extending through a length of said delivery catheter and generally parallel to said guide wire lumen.

7. A stent deployment system as recited in claim 1, wherein said distal end of said at least one wire is attached to an inner surface of said sheath.

8. A stent deployment system for delivering and deploying a stent within a body lumen, comprising:
a delivery catheter having a proximal end, a distal end, an intermediate portion extending between said proximal and distal ends, and a guide wire lumen extending therethrough, said distal end being adapted to have a stent disposed thereon, said intermediate portion comprising at least one slot disposed therein that extends from an outer surface of said intermediate portion to said guide wire lumen;
a sheath adapted to be disposed around said distal end of said delivery catheter to selectively cover the stent disposed on said distal end of said delivery catheter, said sheath being adapted for movement along a length of said delivery catheter to selectively uncover the stent; and
at least one wire having a distal end attached to said sheath to facilitate movement of said sheath along the length of said delivery catheter to selectively uncover the stent, said at least one wire being adapted to pass from said outer surface of said delivery catheter into said guide wire lumen through said at least one slot in said delivery catheter.

9. A stent deployment system as recited in claim 8, wherein said guide wire lumen is adapted to have a guide wire and said at least one wire simultaneously positioned therethrough

10. A stent deployment system as recited in claim 8, wherein said at least one wire has a proximal end adapted to be operatively associated with a retraction mechanism to facilitate proximal movement of said at least one wire.

11. A stent deployment system as recited in claim 10, wherein proximal movement of said at least one wire causes said sheath to be retracted off of the stent.

12. A stent deployment system as recited in claim 8, wherein said at least one slot comprises two slots disposed on opposing sides of said delivery catheter, and said at least one wire comprises two wires, each of said two wires being associated with one of said two slots.

13. A stent deployment system as recited in claim 8, wherein a first portion of said at least one wire is disposed outside said delivery catheter, a second portion of said at least one wire is disposed within said at least one slot, and a third portion of said at least one wire is disposed within said guide wire lumen.

14. A stent deployment system as recited in claim 8, wherein the movement of said sheath along the length of said delivery catheter to selectively uncover the stent enables radial expansion of the stent.

15. A stent deployment system for delivering and deploying a stent within a body lumen, comprising:
a delivery catheter having a proximal end, a distal end, and an intermediate portion extending between said proximal and distal ends, said distal end being adapted to have a stent disposed thereon, said delivery catheter also having at least one retraction wire lumen disposed therein that extends from an opening in an outer surface of said intermediate portion toward said proximal end of said delivery catheter;
a sheath adapted to be disposed around said distal end of said delivery catheter to selectively cover the stent disposed on said distal end of said delivery catheter, said sheath being adapted for movement along a length of said delivery catheter to selectively uncover the stent; and
at least one wire having a distal end attached to said sheath to facilitate movement of said sheath along the length of said delivery catheter to selectively uncover the stent, said at least one wire extending from said sheath, into said opening in said outer surface of said intermediate portion, and through said at least one retraction wire lumen toward said proximal end of said delivery catheter.

16. A stent deployment system as recited in claim 15, wherein said at least one retraction wire lumen extends along a substantial length of said delivery catheter.

17. A stent deployment system as recited in claim 15, wherein a first length of said delivery catheter comprises one lumen and a second length of said delivery catheter comprises at least two lumens.

18. A stent deployment system as recited in claim 17, wherein said one lumen of said first length of said delivery catheter comprises a guide wire lumen, and said at least two lumens of said second length of said delivery catheter comprise said guide wire lumen and said at least one retraction wire lumen.

19. A stent deployment system as recited in claim 15, wherein said opening in said outer surface of said intermediate portion is spaced far enough from said distal end of said delivery catheter to enable said sheath to be positioned entirely between said opening and a proximal end of the stent when said sheath is moved along the length of said delivery catheter to selectively uncover the stent.

20. A stent deployment system as recited in claim 15, wherein the delivery catheter further comprises a stop disposed on the proximal side of the stent to limit longitudinal movement of the stent along the length of the delivery catheter.
